(19) United States
(12) Patent Application Publication
(10) Pub. No.: US 2007/0065485 A1
(43) Pub. Date: Mar. 22, 2007

(54) Therapeutic delivery of carbon monoxide

(76) Inventors: Roberto Angelo Motterlini, Harrow Middlesex (GB); Roger Ariel Alberto, Winterthur (CH)

Correspondence Address: NIXON & VANDERHYE, PC, 901 NORTH GLEBE ROAD, 11TH FLOOR, ARLINGTON, VA, 22203, US

(21) Appl. No.: 10/567,157

(22) PCT Filed: Aug. 4, 2004
(86) PCT No.: PCT/GB04/03365

§ 371 Date: Jun. 1, 2006

(30) Foreign Application Priority Data
Aug. 4, 2003 (GB) 0318254.0
Apr. 27, 2004 (GB) 0409376.1

(51) Int. Cl. A61F 13/00

(52) U.S. Cl. 424/434

(57) ABSTRACT

Boranocarbonates are described for administration to a human or other mammal for delivery of carbon monoxide. The boranocarbonate is a compound or ion adapted to make CO available for physiological effect, and may be administered with a guanylate cyclase stimulant or stabilizer. The physiological effect may be stimulation of neurotransmission, vasodilation or smooth muscle relaxation.

### FIELD OF THE INVENTION

The present invention relates to pharmaceutical compositions and compounds for the therapeutic delivery of carbon monoxide to humans and other mammals. Another use of the composition and compounds is in organ perfusion.

### BACKGROUND OF THE INVENTION

Mammalian cells constantly generate carbon monoxide (CO) gas via the endogenous degradation of heme by a family of constitutive (HO-2) and inducible (HO-1) heme oxygenase enzymes^{1,2}. First described as a putative neural messenger³, CO is now regarded as a versatile signaling molecule having essential regulatory roles in a variety of physiological and pathophysiological processes that take place within the cardiovascular, nervous and immune systems. Indeed, CO produced in the vessel wall by heme oxygenase enzymes possesses vasorelaxing properties and has been shown to prevent vasoconstriction and both acute and chronic hypertension through stimulation of soluble guanylate cyclase ⁴⁻¹⁰. Endogenous CO appears to modulate sinusoidal tone in the hepatic circulation¹¹, control the proliferation of vascular smooth muscle cells¹² and suppress the rejection of transplanted hearts¹³. The biological action of heme oxygenase-derived CO is substantiated by the pharmacological effects observed when this gas is applied exogenously to in vitro and in vivo systems. At concentrations ranging from 10 to 500 p.p.m., CO gas has been reported to mediate potent anti-inflammatory effects¹⁴, prevent endothelial cell apoptosis¹⁵, inhibit human airway smooth muscle cell proliferation¹⁶ and promote protection against hyperoxic as well as ischemic lung injury^{17,18}. In view of the pivotal role exerted by the heme oxygenase pathway in the control of cellular homeostasis¹⁹ and the emerging pleiotropic properties attributed to CO²⁰, it is conceivable that this diatomic gas could be used as a therapeutic tool for the treatment of vascular dysfunction and immuno-related disease states.

At present, three different approaches have been proposed for examining the therapeutic potential of CO: 1) direct administration of CO gas²⁰; 2) use of pro-drugs (i.e. methylene chloride) which are catabolized by hepatic enzymes to generate CO²¹; and 3) transport and delivery of CO by means of specific CO carriers²². Some investigators have concentrated their efforts on the last strategic approach as it has been recently reported that certain transition metal carbonyls possess the ability to liberate CO under appropriate conditions and function as CO-releasing molecules (CO-RMs) in biological systems. In particular, it was shown that CO-RMs induce vessel relaxation in isolated aortic tissue and prevent coronary vasoconstriction as well as acute hypertension in vivo through specific mechanisms that can be simulated by activation of the HO-1/CO pathway²³. Interestingly, the versatile chemistry of transition metals allows them to be effectively modified by coordinating biological ligands to the metal center in order to render the molecule less toxic, more water soluble and to modulate the release of CO. It has been recently reported that tricarbonylchloro(glycinato)ruthenium(II) (here called CORM-3), a newly synthesized water-soluble form of metal carbonyl that liberates CO in vitro, ex-vivo and in vivo biological models, protects myocardial cells and tissues against ischemia-reperfusion injury as well as cardiac allograft rejection^{24,25}. Some of this work is published in International Patent Application WO 02/092075 (ref. 25).

In the case of CORM-3, the chloride and glycinate ligands are labile and their substitution with higher affinity ligands present in the cellular or plasma environment (i.e. glutathione) would appear to accelerate dissociation of CO from the metal center²⁷. When added to a solution containing myoglobin (Mb), the release of CO from CORM-3 is accelerated as 1 mole of CO per mole of compound is liberated within 1-2 min²⁴. CORM-3 would, therefore, fall into a category of compounds that release CO very rapidly (“fast releasers”) which can be ideal for several clinical applications in which CO acts as a signalling mediator (i.e. neurotransmission, acute hypertension, angina, ischemia-reperfusion); however, identifying compounds that release CO with a slow kinetics (“slow releasers”) would implement the design of pharmaceuticals that could be more versatile in the treatment of certain chronic diseases (i.e. arthritis, inflammation, cancer, organ preservation; chronic hypertension; septic shock prevention of restenosis after balloon angioplasty, post-operative ileus) where the continuous and long-lasting effect of CO may be required.

An interesting example in the development of transition metal carbonyls that are used for medical applications not related to the therapeutic use of CO is represented by carbonyls specifically designed for radio-imaging technology. The recently described technetium(I) complex [^{99m}Tc(OH₂)₃—(CO)₃]⁺ has attracted much interest as a precursor for technetium-99m radiopharmaceuticals²⁸. A number of biomolecules, for example, peptides, scFv, and CNS receptor ligands, have already been labeled with technetium by this approach, demonstrating the potential of [^{99m}Tc(OH₂)₃—(CO)₃]⁺ for radiopharmaceutical application²⁹. This compound can be prepared in a single-step procedure from aqueous [^{99m}TcO4]⁻ in the presence of CO and BH₄³¹ as a reducing agent³⁰. However, the published preparation of [^{99m}Tc(OH₂)₃—(CO)₃]⁺ relying on gaseous carbon monoxide, is unsuitable for use in commercial radiopharmaceutical “kits”. A recent study has reported the first commercially feasible preparation of [^{99m}Tc(OH₂)₃—(CO)₃]⁺ in physiological media using a boron-based carbonylating agent, potassium boranocarbonate (K₂[H₃BCO₂]), which acts as a CO source and a reducing agent at the same time³¹.

Boranocarbonates have been disclosed or suggested for physiological effects in the prior art. EP-A-34238 and EP-A-181721 describes anti-tumour and anti-hyperlipidemic activities of amine-carboxboranes. U.S. Pat. No. 4,312,989 discloses use of amine boranes to inhibit the inflammation process. U.S. Pat. No. 5,254,706 describes phosphite-borane compounds for anti-tumour, anti-inflammatory and hypolipidemic activity.

WO93/05795 discusses use of organic boron compounds effective against osteoporosis and suggests also anti-inflammatory, anti-hyperlipidemic and antineoplastic activity. The compounds disclosed are primarily of the amino-borane class, but Na₂BH₃COO is also tested. Hall et al., “Metal Based Drugs”, Vol. 2, No. 1, 1995, describes anti-inflammatory activity of acyclic amine-carboxyboranes in rodents.

These documents reveal interest in the boron compounds either because of the possible effect of boron itself or because the amino-boranes are analogous to the natural α-amino acids.

### SUMMARY OF THE INVENTION

As exemplified by the experimental data detailed below, the present inventors have found that boranocarbonate compounds can be used to deliver CO to a physiological target so as to provide physiological effect.

Accordingly the present invention provides a pharmaceutical composition, intended for administration to a human or other mammal for delivery of carbon monoxide, comprising a boranocarbonate compound or ion adapted to make CO available for physiological effect and at least one pharmaceutically acceptable carrier.

Boranocarbonates are a group of compounds which can loosely be described as carboxylate adducts of borane and derivatives of borane. Boranocarbonates generally contain a group of the form —COO^{—} or COOR (where R is H or another group) attached to the boron atom, so that they may be called boranocarboxylates or carboxyboranes, but the term boranocarbonate seems to be preferred. The compound K₂(H₃BCOO) and the related K(H₃BCOOH) are described in reference 31, where the compound K₂(H₃BCOO) is used for producing Tc carbonyls.

Thus typically a boranocarbonate has the molecular structure including the moiety

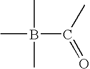

Preferred is the structure above with three hydrogen atoms attached to the boron (BH₃—CO—), since this is believed to facilitate CO release.

Also preferred are structures where a carboxylate group is attached to boron, i.e. —COO⁻, —COOH—, —COOX where X may be any suitable esterifying group acceptable pharmaceutically.

Preferably the boranocarbonate compound in the pharmaceutical composition has an anion of the formula:
BHₓ(COQ)_{y}Z_{z}

wherein:
x is 1, 2 or 3
y is 1, 2 or 3
z is 0, 1 or 2
x+y+z=4,
each Q is O⁻, representing a carboxylate anionic form, or is OH, OR, NH₂, NHR, NR₂, SR or halogen, where the or each R is alkyl (preferably of 1 to 4 carbon atoms),
each Z is halogen, NH₂, NHR′, NR′₂, SR′ or OR′ where the or each R′ is alkyl (preferably of 1 to 4 carbon atoms).
   Since this formula is analogous to the borano anion BH₄⁻, the structure generally is an anion. It may be a divalent anion when one (COQ) is present as (COO⁻). If the structure is an anion, a cation is required. Any physiologically suitable cation may be employed, particularly a metal cation such as an alkali metal ion e.g. K⁺ or Na⁺ or an alkaline earth metal cation such as Ca⁺⁺ or Mg⁺⁺. Alternatively non-metal cations might be employed, such as NR₄⁺ where each R is H or alkyl (preferably of 1 to 4 carbon atoms) or PR₄⁺ where R is alkyl (preferably of 1 to 4 carbon atoms). The cation may be selected in order to achieve a desired solubility of the compound.

Preferably y is 1. Preferably x is 3.

Preferably the boranocarbonate is soluble and is present in solution in a suitable solvent, e.g. an aqueous solvent, in the composition. Other possible solvents are ethanol, DMSO, DMF and other physiologically compatible solvents.

The boranocarbonates employed in the present invention vary in their ability to provide CO. The release of CO may be pH and temperature dependent. Lower pH causes more or faster release. Thus a range of compounds is available, for choice of a suitable release rate for a particular application. Slow release over a long period, of hours or days, can be achieved. Solutions can be provided containing dissolved CO, already released by the boranocarbonate. Alternatively, release of CO may be triggered by change of condition (e.g. pH or temperature) or by contact with another material, e.g. another solvent or aqueous physiological fluid such as blood or lymph, or even at a physiological delivery site.

Typically the pharmaceutical compositions of the present invention release CO such as to make it available to a therapeutic target in dissolved form. However, in some circumstances CO may be released directly to a non-solvent acceptor molecule.

It will be apparent that pharmaceutical compositions according to the present invention may be capable of delivering CO therapeutically through one or more of the above described modes of action.

The boranocarbonate compound may further comprise a targeting moiety, to facilitate release of CO at an appropriate site. The targeting moiety is typically capable of binding a receptor on a particular target cell surface, in order to promote release of CO at the required site. The targeting moiety may be a part of a modulating ligand capable of binding to a receptor found on the surface of the target cells, or may be derived from another molecule, such as an antibody directed against a particular receptor, joined to the boranocarbonate molecule by a suitable linker.

The pharmaceutical compositions of the present invention typically comprise a pharmaceutically acceptable excipient, carrier, buffer, stabiliser or other materials well known to those skilled in the art. Such materials should be non-toxic and should not interfere unduly with the efficacy of the active ingredient. The precise nature of the carrier or other material may depend on the route of administration, e.g. oral, intravenous, subcutaneous, nasal, intramuscular, intraperitoneal, transdermal, transmucosal or suppository routes.

Pharmaceutical compositions for oral administration may be in tablet, capsule, powder or liquid form. A tablet may include a solid carrier such as gelatin or an adjuvant or a slow-release polymer. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Pharmaceutically acceptable amounts of other solvents may also be included, in particular where they are required for dissolving the particular compound contained in the composition.

For intravenous, cutaneous or subcutaneous injection, or injection at the site of affliction, the active ingredient will typically be in the form of a parenterally acceptable solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. Delivery systems for needle-free injection are also known, and compositions for use with such systems may be prepared accordingly.

In pharmaceutical compositions intended for delivery by any route including but not limited to oral, nasal, mucosal, intravenous, cutaneous, subcutaneous and rectal the active substance may be micro encapsulated within polymeric spheres such that exposure to body fluids and subsequent CO release is delayed in time.

Administration is preferably in a prophylactically effective amount or a therapeutically effective amount (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

When formulating pharmaceutical compositions according to the present invention, the toxicity of the active ingredient and/or the solvent must be considered. The balance between medical benefit and toxicity should be taken into account. The dosages and formulations of the compositions will typically be determined so that the medical benefit provided outweighs any risks due to the toxicity of the constituents.

There is further provided a method of introducing CO to a mammal comprising the step of administering a pharmaceutical composition according to the present invention. CO is thought to act at least in part through stimulation or activation of guanylate cyclase. CO is thought to have functions as, inter alia, a neurotransmitter and a vasodilating agent. Accordingly there is provided a method of delivering CO to a mammal for stimulation of guanylate cyclase activity. There is further provided a method of delivering CO to a mammal for stimulating neurotransmission or vasodilation. However the present applicants do not wish to be bound by theory and do not exclude the possibility that CO operates by other mechanisms.

The heme oxygenase 1 (HO-1) pathway is thought to represent a pivotal endogenous inducible defensive system against stressful stimuli including UVA radiations, carcinogens, ischaemia-reperfusion damage, endotoxic shock and several other conditions characterised by production of oxygen free radicals (32, 19, 2). The protective effect of HO-1 is attributed to the generation of the powerful antioxidants biliverdin and bilirubin and the vasoactive gas CO. Expression of HO-1 has been linked with cardiac xenograft survival (33), suppression of transplant arteriosclerosis (34) and amelioration of post-ischemic myocardial dysfunction (35). HO-1 has also been directly implicated in the resolution phase of acute inflammation in rats (36). Other pathological situations, such as haemorrhagic shock in brain and liver as well as sepsis (37-39), are characterized by induction of the HO-1 gene, which seems to play a crucial role in counteracting the vascular dysfunction caused by these pathophysiological states. Increased generation of CO as a consequence of HO-1 induction markedly affects vessel contractility and diminishes acute hypertension in the whole organism (10, 9). Exposure of animals to ambient air containing low concentrations of CO or transfection of the HO-1 gene results in protection against hyperoxia-induced lung injury in vivo, a mechanism mediated by attenuation of both neutrophil inflammation and lung apoptosis (cell death) (17, 40). Exogenous CO gas also has the ability to suppress pro-inflammatory cytokines and modulate the expression of the anti-inflammatory molecule, IL-10, both in vitro and in vivo (14). Therefore administration of CO in accordance with the invention may be used for treatment of any of these conditions, for modulation of inflammatory states and regression of other pathophysiological conditions including cancer.

Accordingly there is provided a method of introducing CO to a mammal comprising the step of administering a pharmaceutical composition according to the present invention, for treatment of hypertension, such as acute, pulmonary and chronic hypertension, radiation damage, endotoxic shock, inflammation, inflammatory-related diseases such as asthma, rheumatoid arthritis and small bowel disease, hyperoxia-induced injury, apoptosis, cancer, transplant rejection, post-operative ileus, arteriosclerosis, post-ischemic organ damage, myocardial infarction, angina, haemorrhagic shock, sepsis, penile erectile dysfunction and adult respiratory distress syndrome, and in procedures such as balloon angioplasty (to treat restenosis following balloon angioplasty) and aortic transplantation. For example, in balloon angioplasty it may be advantageous to make a local delivery of CO-releasing compound before and/or after the angioplasty. Alternatively, a stent may have a coating containing CO- releasing compounds.

The present invention also provides the use of a boranocarbonate compound or ion as herein described in the manufacture of a medicament for delivering CO to a physiological target, particularly a mammal, to provide a physiological effect, e.g. for stimulating neurotransmission or vasodilation, or for treatment of any of hypertension, such as acute, pulmonary and chronic hypertension, radiation damage, endotoxic shock, inflammation, inflammatory-related diseases such as asthma, rheumatoid arthritis and small bowel disease, hyperoxia-induced injury, apoptosis, cancer, transplant rejection, post-operative ileus, arteriosclerosis, sickle cell anemia or sickle cell disease, post-ischemic organ damage, myocardial infarction, angina, haemorrhagic shock, sepsis, penile erectile dysfunction and adult respiratory distress syndrome, and in procedures such as balloon angioplasty and aortic transplantation. Such medicaments may be adapted for administration by an oral, intravenous, subcutaneous, nasal, inhalatory, intramuscular, intraperitoneal, transdermal, transmucosal or suppository route.

In a further aspect, the invention provides a method of treatment of a mammal comprising stimulation of neurotransmission, vasodilation or smooth muscle relaxation by CO as a physiologically effective agent, or the treatment of any of hypertension, radiation damage, endotoxic shock, inflammation, inflammatory-related diseases, hyperoxia-induced injury, apoptosis, cancer, transplant rejection, post-operative ileus, arteriosclerosis, post-ischemic organ damage, myocardial infarction, angina, haemorrhagic shock, sepsis, penile erectile dysfunction, adult respiratory distress syndrome, vascular restenosis, hepatic cirrhosis, cardiac hypertrophy, heart failure and ulcerative colitis, or treatment in balloon angioplasty, aortic transplantation or survival of a transplanted organ, by administration of a boranocarbonate compound or ion adapted to make CO available for physiological effect. These are treatments associated with the action of CO.

Preferably, the method of treatment is stimulation of neurotransmission, vasodilation or smooth muscle relaxation by CO as a physiologically effective agent, or treatment of any of acute or chronic systemic hypertension, radiation damage, endotoxic shock, hyperoxia-induced injury, apoptosis, cancer, transplant rejection, post-operative ileus, arteriosclerosis, post-ischemic organ damage, angina, haemorrhagic shock, sepsis, penile erectile dysfunction, vascular restenosis, hepatic cirrhosis, cardiac hypertrophy, heart failure and ulcerative colitis, or treatment in balloon angioplasty, aortic transplantation or survival of a transplanted organ.

More preferably, the method of treatment is stimulation of neurotransmission, vasodilation or smooth muscle relaxation by CO as a physiologically effective agent, or treatment of any of acute or chronic systemic hypertension, hyperoxia-induced injury, cancer by the pro-apoptotic effect of CO, transplant rejection, post-operative ileus, post-ischemic organ damage, angina, haemorrhagic shock, penile erectile dysfunction, hepatic cirrhosis, cardiac hypertrophy, heart failure and ulcerative colitis, or treatment in balloon angioplasty or aortic transplantation.

Particularly, the method may be treatment of any of hyperoxia-induced injury, cancer by the pro-apoptotic effect of CO, transplant rejection, post-operative ileus, post-ischemic organ damage, angina, haemorrhagic shock, penile erectile dysfunction, hepatic cirrhosis, cardiac hypertrophy, heart failure and ulcerative colitis, or treatment in balloon angioplasty or aortic transplantation.

By “smooth muscle relaxation” is meant treatment of conditions other than by vasodilation, such as chronic anal fissure, internal anal sphincter disease and anorectal disease.

More specific treatments to which the invention may be applied are the suppression of atherosclerotic legions following aortic transplantation, ischemic lung injury, prevention of reperfusion induced myocardial damage, and also to achieve the pro-apoptotic effects of CO (e.g. in cancer treatments).

The invention further provides use of the boranocarbonate compounds or ions here described in treatment, e.g. by perfusion, of a viable mammalian organ extracorporeally, e.g. during storage and/or transport of an organ for transplant surgery. For this purpose, the boranocarbonate is in dissolved form, preferably in an aqueous solution. The viable organ may be any tissue containing living cells, such as a heart, a kidney, a liver, a skin or muscle flap, etc.

For example, isolated organs e.g. extracorporeal organs or in situ organs isolated from the blood supply can be treated. The organ may be, for example, a circulatory organ, respiratory organ, urinary organ, digestive organ, reproductive organ, neurological organ, muscle or skin flap or an artificial organ containing viable cells. In particular, the organ may be a heart, lung, kidney or liver. However, the body tissue which is treatable are not limited and may be any human or mammal body tissue whether extracorporeal or in-situ in the body. It is further believed that the compositions of the invention here described are useful to deliver CO to an extracorporeal or isolated organ so as to reduce ischaemic damage of the organ tissue.

Within the present invention, the boranocarbonates here described can be used in combination with a guanylate cyclase stimulant or stabilizer to deliver CO to a physiological target so as to provide an improved physiological effect.

The pharmaceutical preparation may contain the boranocarbonate and the guanylate cyclase stimulant/stabilizer in a single composition or the two components may be formulated separately for simultaneous or sequential administration.

Thus the present invention provides a method of introducing CO to a mammal as a therapeutic agent comprising:

a) administering a boranocarbonate which makes available CO suitable for physiological effect; and

b) administering a guanylate cyclase stimulant or stabiliser.

In this aspect, the method is particularly applicable to treatment of acute or chronic systemic hypertension, pulmonary hypertension, transplant rejection, post-operative ileus, arteriosclerosis, post-ischemic organ damage, myocardial infarction, penile erectile dysfunction, vascular restenosis, hepatic cirrhosis, cardiac hypertrophy, heart failure, chronic anal fissure, internal anal sphincter disease, anorectal disease, and ulcerative colitis or for treatment in balloon angioplasty or aortic transplantation.

Preferably, the stabilizer/stimulant is administered first followed by the boranocarbonate but this order may be reversed.

The guanylate cyclase stabilizer/stimulant compound may be any compound which stimulates production of guanylate cyclase or which stabilizes guanylate cyclase, in particular the active form of guanylate cyclase. A single compound can be used or a combination of compounds can be used either for simultaneous or sequential administration, i.e. the various aspects include/use at least one guanylate cyclase stimulant/stabilizer.

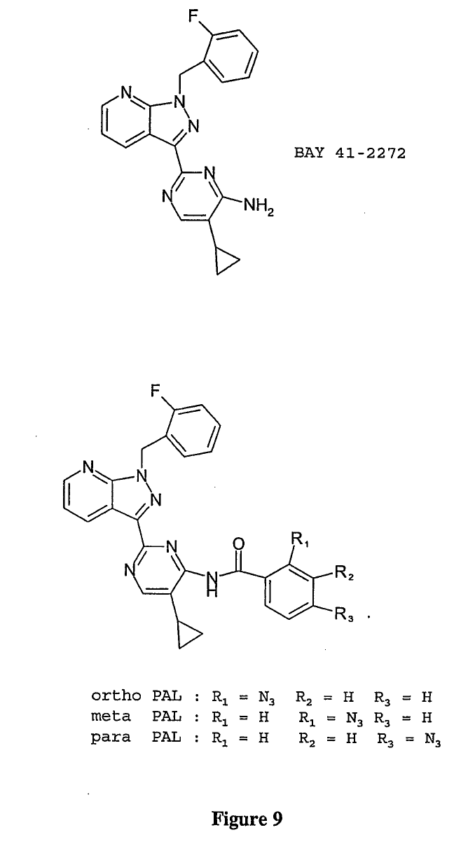

Examples include 3-(5′-hydroxymethyl-2′-furyl)-1-benzyl-indazole (YC-1), 4 pyrimidinamine-5-cyclopropyl-2-[1-[(2-fluorophenyl)methyl]-1H-pyrazolo[3,4-b]pyridin-3-yl] (BAY 41-2272), BAY 50-6038 (ortho-PAL), BAY 51-9491 (meta PAL), and BAY 50-8364 (para PAL). The structures of ortho-, meta- and para- PAL are shown in FIG. 9 attached. These compounds have been found to bind to an activation site on the guanylate cyclase and any other compounds that similarly bind to the site may be useful as the guanylate cyclase stabilizer/stimulant. Also useful are NO donors and 1-benzyl-3-(3¹-ethoxycarbonyl)phenyl-indazole, 1-benzyl-3-(3¹-hydroxymethyl)phenyl-indazole, 1-benzyl-3-(5¹-diethylaminomethyl)-furyl-indazole, 1-benzyl-3-(5¹-methoxymethyl)furyl-indazole, 1-benzyl-3-(5¹-hydroxymethyl)furyl-6-methyl-indazole, 1-benzyl-3-(5¹-hydroxymethyl)-furyl-indazol-benzyl-3-(5¹-hydroxymethyl)-furyl-indazole, 1-benzyl-3-(5¹-hydroxymethyl)-furyl-6-fluoro-indazole, 1-benzyl-3-(5¹-hydroxymethyl)-furyl-6-methoxy-indazole, and 1-benzyl-3-(5¹-hydroxymethyl)-furyl-5,6-methylenedioxoindazole or pharmaceutically acceptable salts thereof.

For reasons relating to prior patent filings and for proprietary reasons, the present applicants may wish to exclude use of the following two compounds from the protection given to the present invention in any of its aspects as claimed:

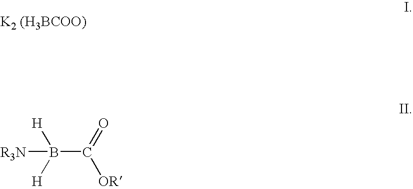

where R, R′═H, alkyl, perfluoroalkyl.

Therefore this exclusion is now optionally and provisionally made.

Throughout this application, references to medical treatment are intended to include both human and veterinary treatment, and references to pharmaceutical compositions are accordingly intended to encompass compositions for use in human or veterinary treatment.

Experimental data illustrating the present invention will now be described.

In the accompanying drawings,

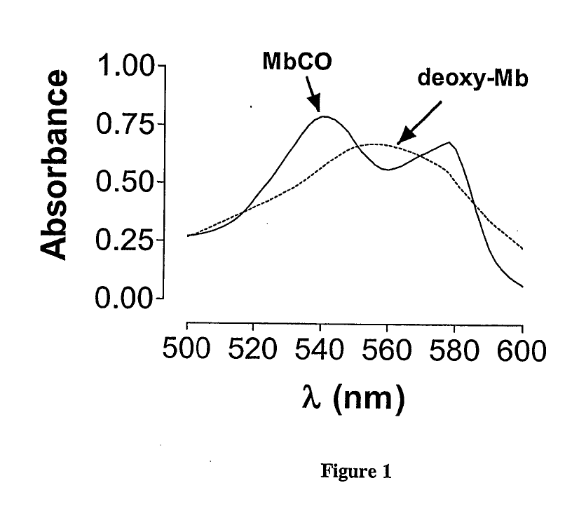

FIGS. 1 to 8 are graphs showing results of the experiments of Examples 1 to 8 below.

FIG. 9 is chemical formulae mentioned above.

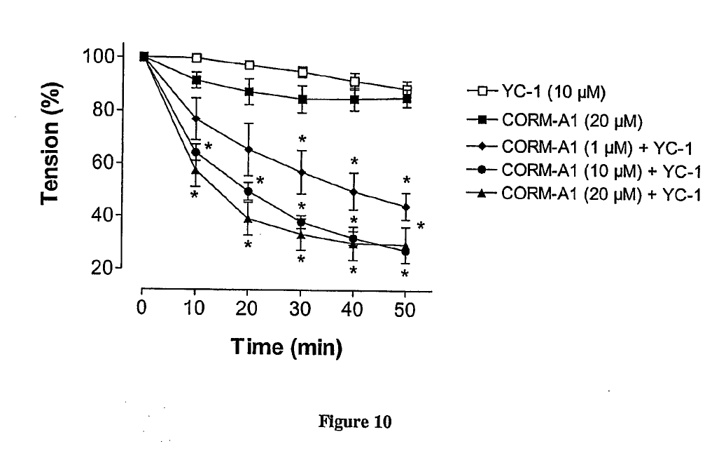

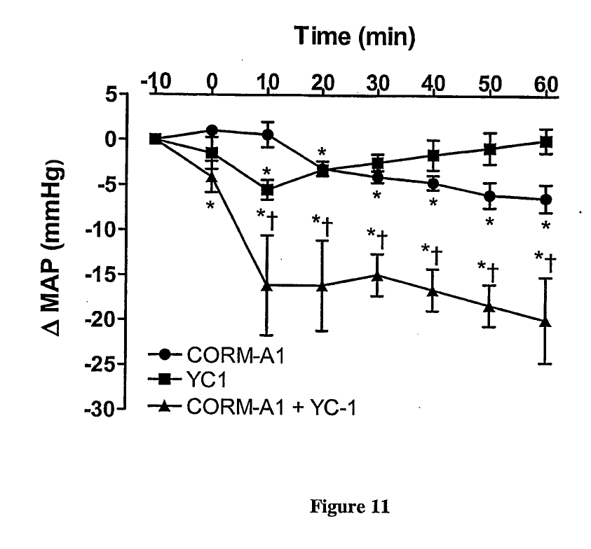

FIGS. 10 and 11 are graphs showing results of Examples 9 and 10 below.

### EXAMPLES 1 TO 8

Reagents

Tricarbonylchloro(glycinato)ruthenium(II) ([Ru(CO)₃Cl(glycinate)] or CORM-3) was synthesized as previously described by Clark and collaborators²⁴. Disodium boranocarbonate (Na₂[H₃BCO₂], indicated here as “CORM-A1”) was synthesized as previously described by Alberto and collaborators³¹. Sodium borohydride (NaBH₄) and all other reagents were from Sigma Chemicals (Poole, Dorset).

Preparation of Inactive CORM-A1 and its Use as Negative Control

The chemistry of boranocarbonate in aqueous solution has been previously described³¹. This compound is relatively stable in distilled water at basic pH. The compound starts to release CO as the pH moves towards more physiological conditions (pH=7.4) and the rate of CO release is greatly accelerated at acidic pH. Based on this evidence, we generated an inactive form of CORM-A1 (iCORM-A1) by reaction of the compound with acid. Specifically, a small aliquot (10 μl) of concentrated hydrochloric acid (10 M) was added to 1 ml of CORM-A1 in water (100 mM final concentration). The reaction resulted in a rapid evolution of a gas (presumably CO); the solution was then bubbled with a stream of nitrogen in order to remove the residual CO gas eventually dissolved. Aliquots of this solution were used as a negative control of CORM-A1 in the experiments conducted to quantify the release of CO (i.e. Mb assay) as well as the biological efficacy (i.e. vessel relaxation). Since boron is a component of CORM-A1 and because borohydride could be formed during the liberation of CO from CORM-A1 in aqueous solution, sodium borohydride (NaBH₄) was also utilized as a negative control in some experiments.

Detection of CO Release

The release of CO from CORM-A1 was assessed spectrophotometrically by measuring the conversion of deoxymyoglobin (Mb) to carbonmonoxy myoglobin (MbCO) by a method previously described²³. The amount of MbCO formed was quantified by measuring the absorbance at 540 nm (extinction coefficient=15.4 M⁻¹ cm⁻¹) over time at 37° C. Myoglobin solutions (approximately 50 μmol/L final concentration) were prepared fresh by dissolving the protein in 0.04 M phosphate buffer (pH=7.4). Sodium dithionite (0.1%) was added to convert the oxidized myoglobin to its reduced form prior to each reading. Some experiments were also conducted using Mb at pH=5.5 or at room temperature (RT) in order to examine the kinetic of CO release from CORM-A1 under different chemical and physical conditions.

Isolated Aortic Ring Preparation: Studies on Vessel Relaxation

Transverse ring sections of thoracic aorta were isolated from male Lewis rats and suspended under a 2 g tension in an organ bath containing oxygenated Krebs-Henseleit buffer at 37° C. in a manner previously described¹⁰. The relaxation response to CORM-A1 (40, 80 and 160 μM) was assessed in aortic rings pre-contracted with phenylephrine (3 μM). Control rings were similarly treated by adding equal doses of the inactive compound (iCORM-A1) or sodium borohydride (NaBH₄) to the organ bath. Experiments were also conducted by comparing the effect of CORM-A1 and CORM-3 on vessel relaxation over time.

### EXAMPLE 1

### Conversion of Myoglobin (Mb) to Carbon Monoxide Myoglobin (MbCO) by CO Gas

Myoglobin (Mb) in its reduced state displays a characteristic spectrum with a maximal absorption peak at 555 nm (see FIG. 1, dotted line). When a solution of Mb (50 μM) is bubbled for 1 min with Co gas (1%), a rapid conversion to carbon monoxide myoglobin (MbCO) is observed. As shown in FIG. 1, MbCO displays a characteristic spectrum with two maximal absorption peaks at 540 and 576 nm, respectively (solid line). This method has been previously developed to monitor and determine the amount of CO released from CO-RMs²³ and can be used to examine how various conditions such as different pHs and temperatures can affect the kinetics of CO release (see Examples 4).

### EXAMPLE 2

### Conversion of Myoglobin (Mb) to Carbon Monoxide Myoglobin (MbCO) by CORM-A1

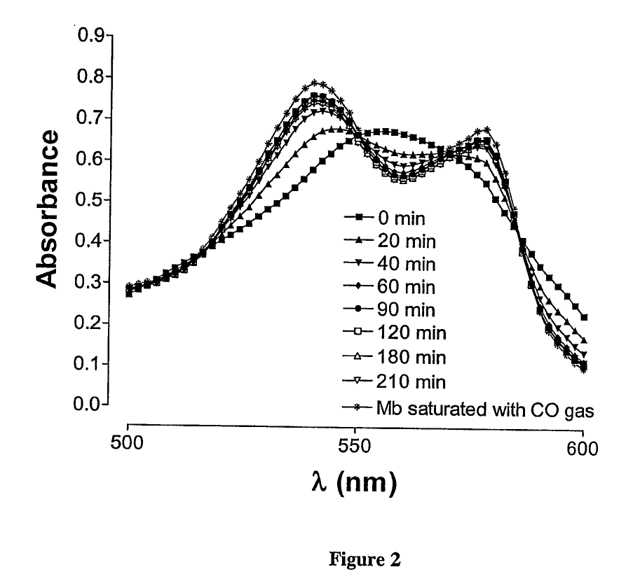

Addition of CORM-A1 (60 μM) to a solution containing reduced Mb (pH=7.4, temp.=37° C.) resulted in a gradual formation of MbCO over time. As shown in FIG. 2, a spectrum typical of reduced Mb (filled square) is converted to a spectrum characteristic of MbCO after 210 min incubation (inverted open triangle). The trace with asterisks shows the spectrum of MbCO when Mb is saturated with CO gas (positive control) as described in Materials and Methods.

### EXAMPLE 3

### Kinetics of Co Release from CORM-A1 at Room Temperature

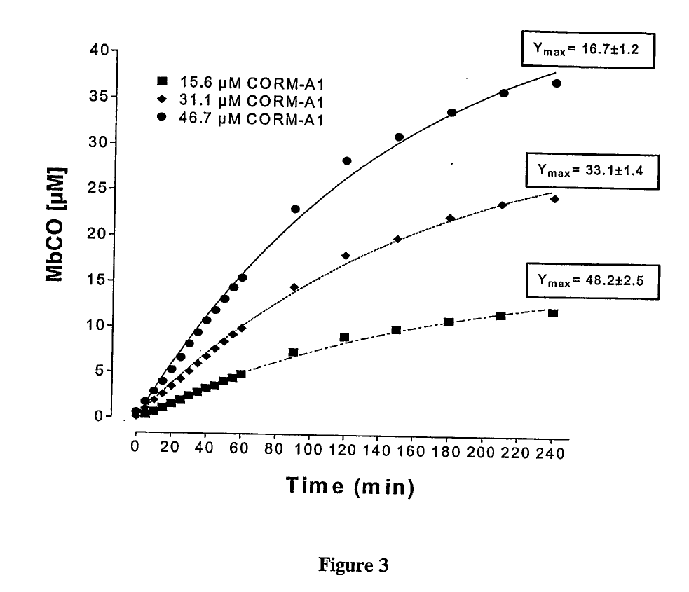

The amount of MbCO formed after addition of CORM-A1 to the Mb solution can be quantified by measuring the absorbance at 540 nm knowing the extinction coefficient for MbCO (ε=15.4 M⁻¹ cm⁻¹). CORM-A1 at three different concentrations was added to a solution containing Mb at room temperature and the formed MbCO was calculated over time. Non-linear regression analysis using one phase exponential association (GraphPad Prism) resulted in the best fitting of the three curves (r²>0.99). As shown in FIG. 3, the amount of MbCO formed from CORM-A1 increases with a defined kinetic in a concentration-dependent manner. The calculated Yₘₐₓ for each plot (16.7±1.2, 33.1±1.4 and 48.2±2.5) was in very good agreement with the three concentrations of CORM-A1 used (15.6, 31.1 and 46.7 μM, respectively). This indicates that the reaction leading to the formation of CO from CORM-A1 in aqueous solution goes to completion over time and that one mole of CO per mole of compound is liberated. From the fitted curves the average half-life of CORM-A1 at room temperature is 112±3 min.

### EXAMPLE 4

### Effects of Temperature and pH on the Rate of CO Release from CORM-A1

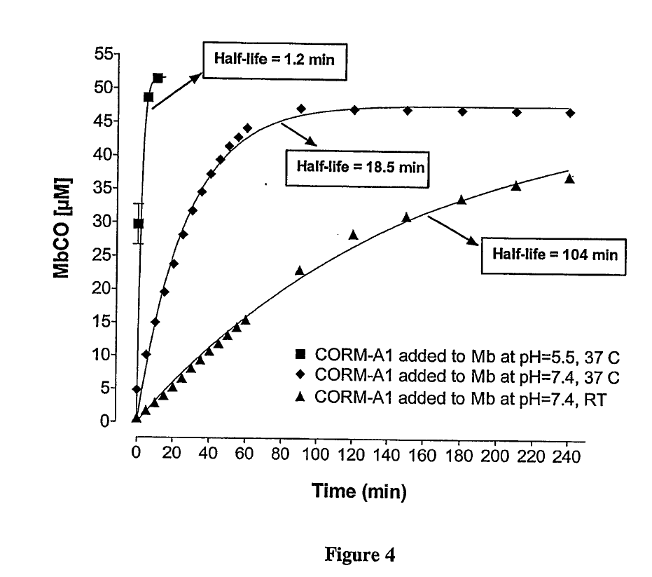

The rate of CO release from CORM-A1 was examined at different pHs and temperatures. CORM-A1 (60 μM) was added to the Mb solution under three different conditions: 1) at room temperature (RT) and pH=7.4; 2) at 37° C. and pH=7.4; and 3) at 37° C. and pH=5.5. The concentration of MbCO was calculated at different time points and non-linear regression analysis was used to obtain the best fitting of the three curves as described in example 3. As shown in FIG. 4, the rate of CO release from CORM-A1 is significantly accelerated by increasing the temperature as well as by decreasing the pH. Specifically, it can be calculated that the half-life of CORM-A1 is 104 min at RT/pH=7.4 (triangles), 18.5 min at 37° C./pH=7.4 (diamonds) and 1.2 min at 37° C./pH=5.5 (squares).

### EXAMPLE 5

### Comparison between CORM-AL and its Inactive Form (iCORM-A1) on their Ability to Liberate CO

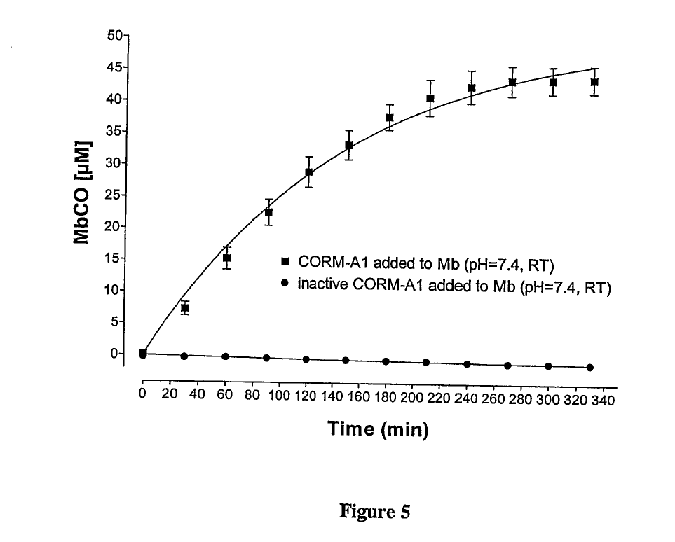

As described in the Materials and Methods section, CO is rapidly lost when CORM-A1 is added to acidic solutions. This step allows the generation of an inactive compound (iCORM-A1) that could be used as an ideal negative control for testing the biological activity of these molecules. To verify that iCORM-A1 has effectively lost its full ability to release CO, the compound (60 μM) was added to a solution containing Mb (50 μM) at pH=7.4/RT and the MbCO formed over time was calculated. As shown in FIG. 5, iCORM-A1 (circles) is incapable of generating any detectable MbCO suggesting that the compound has been fully inactivated. The effect of CORM-A1 (squares) on MbCO formation is shown for comparison.

### EXAMPLE 6

### Comparison between CORM-A1 and CORM-3 in their Ability to Elicit Vasorelaxation

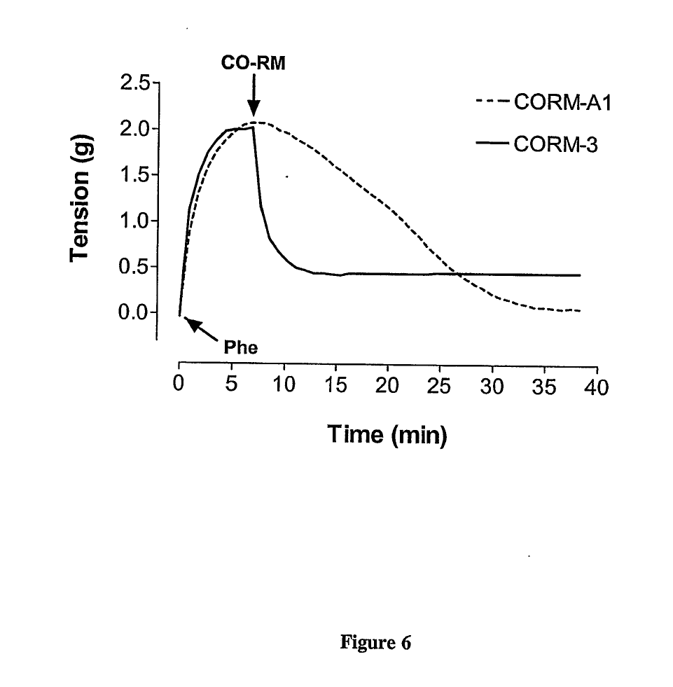

CORM-3 ([Ru(CO)₃Cl(glycinate)]) has been shown to promote a rapid and significant relaxation in isolated vessels and this effect has been demonstrated to be mediated by Co²⁷. It is also known from recent works that the liberation of CO from CORM-3 to Mb or in biological systems occurs very rapidly (approximately 5 min)^{24,27}, which is in agreement with the prompt pharmacological effects observed in isolated vessels. In the case of CORM-A1, the release of CO at physiological pH is slower (18.4 min) as shown in example 5. Thus, it is expected that the pharmacological action of CORM-A1 would reflect-its biochemical behaviour. Indeed, as shown in FIG. 6, CORM-A1 (80 μM) caused a much slower effect on relaxation compared to CORM-3 (80 μM). Specifically, CORM-3 (solid line) added to isolated aortic rings pre-contracted with phenylephrine (Phe) promoted a 75% relaxation within 4-5 min whereas CORM-A1 (dashed line) caused a gradual vasorelaxation which was maximal (96%) 33 min following addition of the compound to the organ bath.

### EXAMPLE 7

### Concentration-Dependent Effect of CORM-A1 on Vasorelaxation

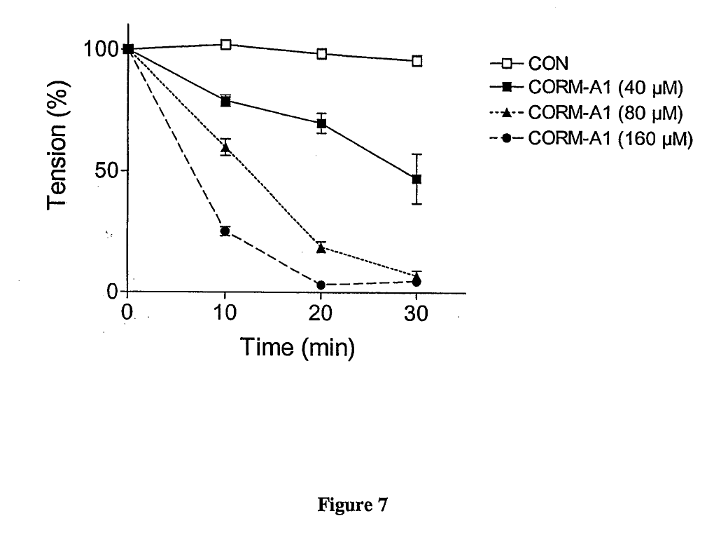

Pre-contracted aortic rings were treated with increasing concentrations of CORM-A1 (40, 80 and 160 μM) and the percentage of vasorelaxation was calculated at different time points. As shown in FIG. 7, CORMA-1 caused a significant relaxation over time in a concentration-dependent manner. For instance, it can be seen from the graph that after 10 min, the percentage of relaxation elicited by the different concentrations of CORM-A1 compared to control was as follows: 21.0±2.3% with 40 μM CORM-A1, 40.2±3.4% with 80 μM CORM-A1 and 74.9±1.8% with 160 μM CORM-A1. The data are represented as the mean±S.E.M. of 6 independent experiments for each group.

### EXAMPLE 8

### The Vasorelaxant Properties of CORM-A1 are Mediated by CO

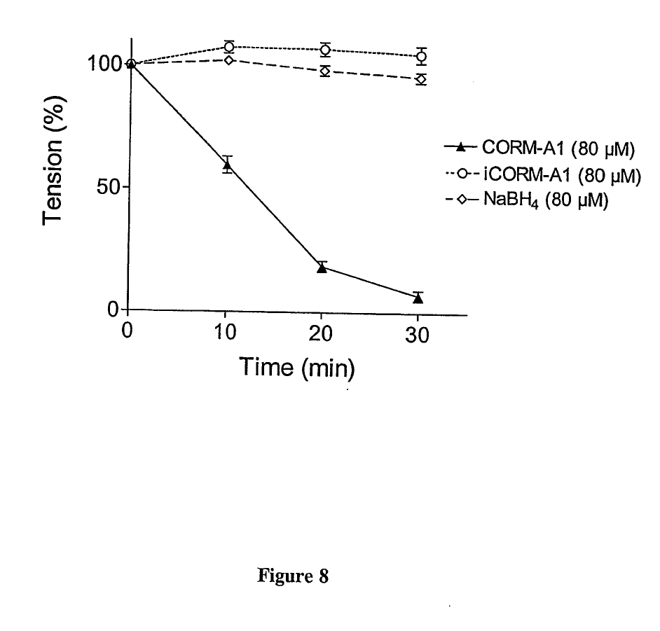

Pre-contracted aortic rings were treated with 80 μM CORM-A1, iCORM-A1 (the inactive compound) or NaBH₄, which was used as an additional negative control (see Materials and Methods for details). As shown in FIG. 8, only CORM-A1 promoted a gradual and profound vasorelaxation whereas both iCORM-A1 and NaBH₄ were totally ineffective. These results clearly suggest that CO liberated from CORM-A1 is directly responsible for the observed pharmacological effect. The data are represented as the mean±S.E.M. of 6 independent experiments for each group.

### EXAMPLE 9 AND 10

Stock solutions of sodium boranocarbonate (CORM-A1, 100 mM) were prepared by solubilizing the compound in distilled water prior to the experiment. 3-(5′-hydroxymethyl-2′-furyl)-1-benzyl-indazole (YC-1) was purchased from Sigma-Aldrich (Poole, Dorset) and prepared in dimethyl sulfoxide (DMSO). All data are expressed as mean±s.e.m. Differences between the groups analysed were assessed by the Student's two-tailed t-test, and an analysis of variance (ANOVA) was performed where more than two treatments were compared. Results were considered statistically significant at P<0.05.

Isolated Aortic Ring Preparation: Studies on Vessel Relaxation

Transverse ring sections of thoracic aorta were isolated from male Lewis rats and suspended under a 2 g tension in an organ bath containing oxygenated Krebs-Henseleit buffer at 37° C. in a manner previously described [10]. The relaxation response to CORM-A1 (20 μM) in the presence or absence of YC-1 (1 μM final concentration) was assessed over time in aortic rings pre-contracted with phenylephrine (1 μmol/L). YC-1 was added to the isolated rings 30 min prior to contraction with phenylephrine.

Animal studies: Effect of CORM-A1 and YC-1 on Blood Pressure

Lewis rats (280-350 g) were anaesthetised by intramuscular injection of 1 ml/kg Hypnorm. Specially designed femoral artery and venous catheters were then surgically implanted and mean arterial pressure (MAP) monitored continuously using a polygraph recorder in a manner previously described [23]. The effect of CORM-A1 on mean arterial pressure (MAP) over time was assessed following an intravenous (i.v.) injection of 50 μmol kg⁻¹. Similar experiments were conducted by administering YC-1 (1.2 μmol kg⁻¹, i.v.) to animals 5 min prior to the bolus addition of CORM-A1. Control experiments using YC-1 alone were also performed.

### EXAMPLE 9

### Effect of CORM-A1 and YC-1 on Aortic Vasorelaxation

Pre-contracted aortic rings were treated with CORM-A1 and the percentage of vasorelaxation was calculated at different time points. As shown in FIG. 10, 20 μM CORMA-1 caused 13±4.9% relaxation after 20 min; interestingly, a more pronounced and significant relaxation response (61±6.2%) was detected after pre-treatment of vessels with YC-1 (1 μM). Note that in control vessels pre-treated with YC-1 alone and contracted with phenylephrine there was only a minor relaxation response over time (2.8±1.1% after 20 min). The relaxation response of vessels pre-treated with YC-1 was also very significant at 1 μM and 10 μM CORM-A1 (35±9.8% and 51±3.3%, respectively). The data are represented as the mean±s.e.m. of 6 independent experiments for each group. *P<0.05 vs. CORM-A1 alone or YC-1 alone.

### EXAMPLE 10

### Effect of CORM-A1 and CORM-3 on Mean Arterial Pressure

Femoral artery and venous catheters were surgically implanted into anesthetized Lewis rats and blood pressure continuously monitored as previously described by us [23]. The effect of CORM-A1 and YC-1 on mean arterial pressure (MAP) in vivo is represented in FIG. 11. The compounds were injected intravenously as a bolus at a final concentration of 50 μmoles/kg for CORM-A1 and 1.2 μmol kg⁻¹ for YC-1. When the two compounds were given in combination, YC-1 was administered 10 min prior to CORM-A1 injection. As shown, CORM-A1 produced a gradual and sustained decrease in MAP over time; for instance, 60 min after CORM-A1 injection MAP decreased by 6.3±1.5 mmHg from the initial baseline value. Injection with YC-1 alone also produced an effect on blood pressure; however, the decrease in MAP was only transient, reaching a maximum of 5.5±1.0 mmHg after 10 min and returning to basal levels 50 min after injection. Interestingly, the combination of CORM-A1 and YC-1 produced a synergistic effect resulting in a rapid and profound hypotension. In fact, MAP significantly decreased by 16.1±5.6 mmHg after 10 min and remained at this level for the rest of the experiment. The data are represented as the mean±s.e.m. of 5 independent experiments for each group. *P<0.05 vs. baseline (−10 min); ^{⇑}P<0.05 vs. CORM-A1 alone or YC-1 alone.

The present invention therefore provides water-soluble compounds which are useful as CO carriers which can have selectable chemical properties, enabling novel therapeutic approaches based on CO delivery. This offers significant advantages over inhalation of CO as it may circumvent the problems related to the systemic effects of CO gas on oxygen transport and delivery. Moreover, the design of stable compounds with “fast” or “slow” kinetics of CO release that could target selective organs and affect only a restricted area of the body is highly feasible. One application for the use of water-soluble compounds is in conditions where Co needs to be applied locally. For instance, in order to protect vascular tissues during balloon angioplasty and prevent blood vessel restenosis, CO-providing compounds may be applied to vessels prior to the angioplasty procedure. Alternatively, vascular stents may be covered with specific boranocarbonate compounds that have the ability to release CO slowly to the injured vessels and inhibit smooth muscle cell proliferation. Compounds whose kinetic of CO release is affected by temperature could also be used ex-vivo as an adjuvant to preservation solutions that are commonly employed to store organs prior to transplantation. The protective role of HO-1 against organ rejection has been extensively reported and the concept of treating the organ(s) rather than the recipient(s) will have much benefit in the clinical setting of transplantation.

### REFERENCES

1. Tenhunen R, Marver H S, Schmid R. Microsomal heme oxygenase. Characterization of the enzyme. J Biol Chem. 1969; 244:6388-6394.
2. Maines M D. The heme oxygenase system: a regulator of second messenger gases. Annu Rev Pharmacol Toxicol. 1997; 37:517-554.
3. Verma A, Hirsch D J, Glatt C E, Ronnett G V, Snyder S H. Carbon monoxide: a putative neural messenger. Science. 1993; 259:381-384.
4. Sacerdoti D, Escalante B, Abraham N G, McGiff J C, Levere R D, Schwartzman M L. Treatment with tin prevents the development of hypertension in spontaneously hypertensive rats. Science. 1989; 243:388-390.
5. Marks G S, Brien J F, Nakatsu K, McLaughlin B E. Does carbon monoxide have a physiological function? Trends Pharmacol Sci. 1991; 12:185-188.
6. Coceani F, Kelsey L, Seidlitz E, Marks G S, McLaughlin B E, Vreman H J, Stevenson D K, Rabinovitch M, Ackerley C. Carbon monoxide formation in the ductus arteriosus in the lamb: implications for the regulation of muscle tone. Br J Pharmacol. 1997; 120:599-608.
7. Johnson R A, Colombari E, Colombari D S A, Lavesa M, Talman W T, Nasjletti A. Role of endogenous carbon monoxide in central regulation of arterial pressure. Hypertension. 1997; 30:962-967.
8. Wang R, Wang Z Z, Wu L Y. Carbon monoxide-induced vasorelaxation and the underlying mechanisms. Br J Pharmacol. 1997; 121:927-934.
9. Motterlini R, Gonzales A, Foresti R, Clark J E, Green C J, Winslow R M. Heme oxygenase-1-derived carbon monoxide contributes to the suppression of acute hypertensive responses in vivo. Circ Res. 1998; 83:568-577.
10. Sammut I A, Foresti R, Clark J E, Exon D J, Vesely M J J, Sarathchandra P, Green C J, Motterlini R. Carbon monoxide is a major contributor to the regulation of vascular tone in aortas expressing high levels of haeme oxygenase-1. Br J Pharmacol. 1998; 125:1437-1444.
11. Suematsu M, Goda N, Sano T. Kashiwagi S, Egawa T, Shinoda Y, Ishimura Y. Carbon monoxide: an endogenous modulator of sinusoidal tone in the perfused rat liver. J Clin Invest. 1995; 96:2431-2437.
12. Morita T, Mitsialis S A, Koike H, Liu Y X, Kourembanas S. Carbon monoxide controls the proliferation of hypoxic vascular smooth muscle cells. J Biol Chem. 1997; 272:32804-32809.
13. Sato K, Balla J, Otterbein L, Smith R N, Brouard S, Lin Y, Csizmadia E, Sevigny J, Robson S C, Vercellotti G, Choi A M, Bach F H, Soares M P. Carbon monoxide generated by heme oxygenase-1 suppresses the rejection of mouse-to-rat cardiac transplants. J Immunol. 2001; 166:4185-4194.
14. Otterbein L E, Bach F H, Alam J, Soares M, Tao Lu H, Wysk M, Davis R J, Flavell R A, Choi A M. Carbon monoxide has anti-inflammatory effects involving the mitogen-activated protein kinase pathway. Nat Med. 2000; 6:422-8.
15. Brouard S, Otterbein L E, Anrather J, Tobiasch E, Bach F H, Choi A M, Soares M P. Carbon monoxide generated by heme oxygenase 1 suppresses endothelial cell apoptosis. J Exp Med. 2000; 192:1015-1026.
16. Song R, Mahidhara R S, Liu F, Ning W, Otterbein L E, Choi A M. Carbon monoxide inhibits human airway smooth muscle cell proliferation via mitogen-activated protein kinase pathway. Am J Respir Cell Mol Biol. 2002; 27:603-610.
17. Otterbein L E, Mantell L L, Choi A M K. Carbon monoxide provides protection against hyperoxic lung injury. Am J Physiol. 1999; 276:L688-L694.
18. Fujita T, Toda K, Karimova A, Yan S F, Naka Y, Yet S F, Pinsky D J. Paradoxical rescue from ischemic lung injury by inhaled carbon monoxide driven by derepression of fibrinolysis. Nat Med. 2001; 7:598-604.
19. Foresti R, Motterlini R. The heme oxygenase pathway and its interaction with nitric oxide in the control of cellular homeostasis. Free Rad Res. 1999; 31:459-475.
20. Otterbein L E. Carbon monoxide: innovative anti-inflammatory properties of an age-old gas molecule. Antioxid Redox Signal. 2002; 4:309-319.
21. Chauveau C, Bouchet D, Roussel J C, Mathieu P, Braudeau C, Renaudin K, Tesson L, Soulillou J P, Iyer S, Buelow R, Anegon I. Gene transfer of heme oxygenase-1 and carbon monoxide delivery inhibit chronic rejection. Am J Transplant. 2002; 2:581-592.
22. Motterlini R, Foresti R, Green C J. Studies on the development of carbon monoxide-releasing molecules: potential applications for the treatment of cardiovascular dysfunction. In: Carbon Monoxide and Cardiovascular Functions. Wang R, ed. 2002. CRC Press, Boca Raton, Fla.
23. Motterlini R, Clark J E, Foresti R, Sarathchandra P, Mann B E, Green C J. Carbon monoxide-releasing molecules: characterization of biochemical and vascular activities. Circ Res. 2002; 90:E17-E24.
24. Clark J E, Naughton P, Shurey S, Green C J, Johnson T R, Mann B E, Foresti R, Motterlini R. Cardioprotective actions by a water-soluble carbon monoxide-releasing molecule. Circ Res. 2003; 93:e2-e8.
25. Mann B E, Motterlini R. Therapeutic delivery of carbon monoxide. PCT. 2002; WO 02092075.
26. Johnson T R, Mann B E, Clark J E, Foresti R, Green C J, Motterlini R. Metal carbonyls: a new class of pharmaceuticals? Angew Chem Int Ed Engl. 2003; In press.
27. Motterlini R, Mann B E, Johnson T R, Clark J E, Foresti R, Green C J. Bioactivity and pharmacological actions of carbon monoxide-releasing molecules. Curr Pharm Des. 2003; In press.
28. Waibel R, Alberto R, Willuda J, Finnern R, Schibli R, Stichelberger A, Egli A, Abram U, Mach J P, Pluckthun A, Schubiger P A. Stable one-step technetium-99m labeling of His-tagged recombinant proteins with a novel Tc(I)-carbonyl complex. Nat Biotechnol. 1999; 17:897-901.
29. Egli A, Alberto R, Tannahill L, Schibli R, Abram U, Schaffland A, Waibel R, Tourwe D, Jeannin L, Iterbeke K, Schubiger P A. Organometallic 99mTc-aquaion labels peptide to an unprecedented high specific activity. J Nucl Med. 1999; 40:1913-1917.
30. Alberto R, Schibli R, Egli A, Schubiger A P, Abram U, Kaden T A. A novel organometallic aqua complex of technetium for the labeling of biomolecules: Synthesis of [Tc-99m(OH2)(3)(CO)(3)](+) from [(TcO4)-Tc-99m](−) in aqueous solution and its reaction with a bifunctional ligand. J Am Chem Soc. 1998; 120:7987-7988.
31. Alberto R, Ortner K, Wheatley N, Schibli R, Schubiger A P. Synthesis and properties of boranocarbonate: a convenient in situ CO source for the aqueous preparation of [(99m)Tc(OH(2))3(CO)3]+. J Am Chem Soc. 2001; 123:3135-3136.
32. Abraham N G, Drummond G S, Lutton J D, Kappas A. The biological significance and physiological role of heme oxygenase. Cell Physiol Biochem 1996; 6:129-68.
33. Soares M P, Lin Y, Anrather J, Csizmadia E, Takigami K, Sato K, Grey S T, Colvin R P, Choi A M, Poss K D, et al. Expression of heme oxygenase-1 can determine cardiac xenograft survival. Nature Med 1998; 4:1073-7.
34. Hancock W W, Buelow R, Sayegh M H, Turka L A. Antibody-induced transplant arteriosclerosis is prevented by graft expression of anti-oxidant and anti-apoptotic genes. Nature Med 1998; 4:1392-6.
35. Clark J E, Foresti R, Sarathchandra P, Kaur H, Green C J, Motterlini R. Heme oxygenase-1-derived bilirubin ameliorates post-ischemic myocardial dysfunction. Am J Physiol Heart Circ Physiol 2000; 278:H643-51.
36. Willis D, Moore A R, Frederick R, Willoughby D A. Heme oxygenase: a novel target for the modulation of inflammatory response. Nature Med 1996; 2:87-90.
37. Bauer M, Pannen B H J, Bauer I, Herzog C, Wanner G A, Hanselmann R, Zhang J X, Clemens M G, Larsen R. Evidence for a functional-link between stress-response and vascular control in hepatic portal circulation. Am J Physiol 1996; 271:G929-35.
38. Fukuda K, Panter S S, Sharp F R, Noble L J. Induction of heme oxygenase-1 (HO-1) after traumatic brain injury in the rat. Neurosci Lett 1995; 199:127-30.
39. Yet S F, Pellacani A, Patterson C, Tan L, Folta S C, Foster L, Lee W S, Hsieh C M, Perrella M A. Induction of heme oxygenase-1 expression in vascular smooth muscle cells. A link to endotoxic shock. J Biol Chem 1997; 272:4295-301.
40. Otterbein L E, Kolls J K, Mantell L L, Cook J L, Alam J, Choi A M K. Exogenous administration of heme oxygenase-1 by gene transfer provides protection against hyperoxia-induced lung injury. J Clin Invest 1999; 103:1047-54.

1. A pharmaceutical composition comprising a boranocarbonate compound or ion, for the stimulation of neurotransmission, vasodilation or smooth muscle relaxation by CO as a physiologically effective agent, or for the treatment of any of acute or chronic systematic hypertension, radiation damage, endotoxic shock, hyperoxia-induced injury, apoptosis, cancer, transplant rejection, post-operative ileus, arteriosclerosis, post-ischemic organ damage, angina, haemorrhagic shock, sepsis, penile erectile dysfunction, vascular restenosis, hepatic cirrhosis, cardiac hypertrophy, heart failure and ulcerative colitis or for treatment in balloon angioplasty, aortic transplantation or survival of a transplanted organ.

2. A pharmaceutical composition according to claim 1 for the stimulation of neurotransmission, vasodilation or smooth muscle relaxation by CO as a physiologically effective agent, or for the treatment of any of acute or chronic systematic hypertension, hyperoxia-induced injury, cancer by the pro-apoptotic effect of CO, transplant rejection, post-operative ileus, post-ischemic organ damage, angina, haemorrhagic shock, penile erectile dysfunction, hepatic cirrhosis, cardiac hypertrophy, heart failure and ulcerative colitis or for treatment in balloon angioplasty or aortic transplantation.

3. A pharmaceutical composition according to claim 1 suitable for administration by an oral, intravenous, subcutaneous, nasal, inhalatory, intramuscular, intraperitoneal, transdermal, transmucosal or suppository route.

4. A pharmaceutical composition according to claim 1 wherein the molecular structure of the boranocarbonate compound or ion includes the moiety

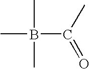

5. A pharmaceutical composition according to claim 4 wherein the boranocarbonate compound or ion includes the moiety BH₃—CO—.

6. A pharmaceutical composition according to claim 4 wherein the boranocarbonate is a compound or anion of the formula:
BHₓ(COQ)_{y}Z_{z}
wherein:
x is 1, 2 or 3
y is 1, 2 or 3
z is 0, 1 or 2
x+y+z=4,
each Q is O⁻, representing a carboxylate anionic form, or is OH, OR, NH₂, NHR, NR₂, SR or halogen, where the or each R is alkyl (preferably of 1 to 4 carbon atoms),
each Z is halogen, NH₂, NHR′, NR′₂, SR′ or OR′ where the or each R′ is alkyl (preferably of 1 to 4 carbon atoms).

7. A pharmaceutical composition according to claim 6 wherein z is 0.

8. A pharmaceutical composition according to claim 6 or 7 where y is 1.

9. A pharmaceutical composition according to claim 6 where x is 3.

10. A pharmaceutical composition according to claim 6 where the boranocarbonate is an anion, with at least one Q in the form of O⁻ or OR, and the composition includes at least one metal cation.

11. A pharmaceutical composition according to claim 10 wherein the or each metal cation is an alkali metal cation or an alkaline earth metal cation.

12. A pharmaceutical composition according to claim 11 wherein the boranocarbonate is Na₂(H₃BCO₂).

13. A pharmaceutical composition according to claim 1 wherein the medicament further includes a guanylate cyclase stimulant or stabilizer.

14. A pharmaceutical composition according to claim 13 wherein the guanylate cyclase stimulant or stabilizer is a molecule or ion uncombined with the boranocarbonate compound or ion.

15. A pharmaceutical composition according to claim 13 wherein the guanylate cyclase stimulant or stabilizer is YC-1.

16. A pharmaceutical composition according to claim 13 wherein the medicament is adapted for one of simultaneous and sequential administration of the boranocarbonate compound or ion and the guanylate cyclase stimulant or stabilizer.

17. A pharmaceutical composition according to claim 1 wherein the boranocarbonate compound or ion is other than 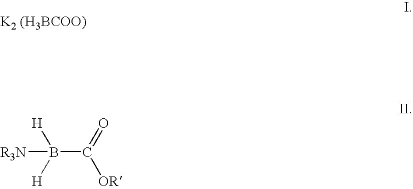
where R, R′═H, alkyl, perfluoroalkyl.

18. Method of treatment of a mammal comprising stimulation of neurotransmission, vasodilation or smooth muscle relaxation by CO as a physiologically effective agent, or the treatment of any of acute or chronic systemic hypertension, radiation damage, endotoxic shock, hyperoxia-induced injury, apoptosis, cancer, transplant rejection, post-operative ileus, arteriosclerosis, post-ischemic organ damage, angina, haemorrhagic shock, sepsis, penile erectile dysfunction, vascular restenosis, hepatic cirrhosis, cardiac hypertrophy, heart failure and ulcerative colitis, or treatment in balloon angioplasty, aortic transplantation or survival of a transplanted organ, by administration of a boranocarbonate compound or ion adapted to make CO available for physiological effect.

19. Method according to claim 18 comprising stimulation of neurotransmission, vasodilation or smooth muscle relaxation by CO as a physiologically effective agent, or treatment of any of acute or chronic systemic hypertension, hyperoxia-induced injury, cancer by the pro-apoptotic effect of CO, transplant rejection, post-operative ileus, post-ischemic organ damage, angina, haemorrhagic shock, penile erectile dysfunction, hepatic cirrhosis, cardiac hypertrophy, heart failure and ulcerative colitis, or treatment in balloon angioplasty or aortic transplantation.

20. Method according to claim 19 wherein including administration by an oral, intravenous, subcutaneous, nasal, inhalatory, intramuscular, intraperitoneal, transdermal, transmucosal or suppository route.

21. Method according to claim 19 wherein the molecular structure of the boranocarbonate compound or ion includes the moiety

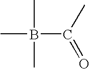

22. Method according to claim 21 wherein the boranocarbonate compound or ion includes the moiety BH₃—CO—.

23. Method according to claim 21 wherein the boranocarbonate is a compound or anion of the formula:
BHₓ(COQ)_{y}Z_{z}
wherein:
x is 1, 2 or 3
y is 1, 2 or 3
z is 0, 1 or 2
x+y+z=4,
each Q is O⁻, representing a carboxylate anionic form, or is OH, OR, NH₂, NHR, NR₂, SR or halogen, where the or each R is alkyl (preferably of 1 to 4 carbon atoms),
each Z is halogen, NH₂, NHR′, NR′₂, SR′ or OR′ where the or each R′ is alkyl (preferably of 1 to 4 carbon atoms).

24. Method according to claim 23 wherein z is 0.

25. Method according to claim 23 where y is 1.

26. Method according to claim 23 where x is 3.

27. Method according to claim 23 where the boranocarbonate is an anion, with at least one Q in the form of O^{- or OR, and the composition includes at least one metal cation.}

28. Method according to claim 27 wherein the or each metal cation is an alkali metal cation or an alkaline earth metal cation.

29. Method according to claim 27 wherein the boranocarbonate is Na₂(H₃BCO₂).

30. Method according to claim 19 wherein the medicament further includes a guanylate cyclase stimulant or stabilizer.

31. Method according to claim 30 wherein the guanylate cyclase stimulant or stabilizer is a molecule or ion uncombined with the boranocarbonate compound or ion.

32. Method according to claim 30 wherein the guanylate cyclase stimulant or stabilizer is YC-1.

33. Method according to claim 30 comprising simultaneous or sequential administration of the boranocarbonate compound or ion and the guanylate cyclase stimulant or stabilizer.

34. Method according to claim 19 wherein the boranocarbonate compound or ion is other than 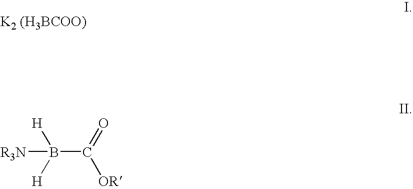
where R, R′═H, alkyl, perfluoroalkyl.

35. A method of treating a viable mammalian organ extracorporeally or an isolated mammalian organ, comprising contacting the organ with a pharmaceutical composition comprising a boranocarbonate compound or ion adapted to make CO available for physiological effect.

36. A method according to claim 35 wherein the boranocarbonate compound or ion is as defined in claim 4.

37. Method according to claim 35 wherein the composition further includes a guanylate cyclase stimulant or stabilizer.

38. Method according to claim 37 wherein the guanylate cyclase stimulant or stabilizer is a molecule or ion uncombined with the boranocarbonate compound or ion.

39. Method according to claim 37 wherein the guanylate cyclase stimulant or stabilizer is YC-1.

40. A medical or veterinary implant carrying, in a form releasable at the implant site, a boranocarbonate compound or ion adapted to make CO available for physiological effect.

41. An implant according to claim 40 wherein the boranocarbonate compound or ion is as defined above.

42. An implant according to claim 40 wherein the medicament further includes a guanylate cyclase stimulant or stabilizer.

43. An implant according to claim 42 wherein the guanylate cyclase stimulant or stabilizer is a molecule or ion uncombined with the boranocarbonate compound or ion.

44. An implant according to claim 42 wherein the guanylate cyclase stimulant or stabilizer is YC-1.

45. A method of introducing CO to a mammal as a therapeutic agent comprising:
a) administering a boranocarbonate which makes available CO suitable for physiological effect; and
b) administering a guanylate cyclase stimulant or stabiliser.

46. A method according to claim 45, which is for the stimulation of neurotransmission, vasodilation or smooth muscle relaxation by CO as a physiologically effective agent, or for the treatment of any of hypertension, radiation damage, endotoxic shock, inflammation, inflammatory-related diseases, hyperoxia-induced injury, apoptosis, cancer, transplant rejection, post-operative ileus, arteriosclerosis, post-ischemic organ damage, myocardial infarction, angina, haemorrhagic shock, sepsis, penile erectile dysfunction, adult respiratory distress syndrome, vascular restenosis, hepatic cirrhosis, cardiac hypertrophy, heart failure and ulcerative colitis or for treatment in balloon angioplasty, aortic transplantation or survival of a transplanted organ.

47. A method according to claim 45, which is for the stimulation of neurotransmission, vasodilation or smooth muscle relaxation by CO as a physiologically effective agent, or for the treatment of any of acute or chronic systematic hypertension, radiation damage, endotoxic shock, hyperoxia-induced injury, apoptosis, cancer, transplant rejection, post-operative ileus, arteriosclerosis, post-ischemic organ damage, angina, haemorrhagic shock, sepsis, penile erectile dysfunction, vascular restenosis, hepatic cirrhosis, cardiac hypertrophy, heart failure and ulcerative colitis or for treatment in balloon angioplasty, aortic transplantation or survival of a transplanted organ.

48. A method according to claim 45, which for the stimulation of neurotransmission, vasodilation or smooth muscle relaxation by CO as a physiologically effective agent, or for the treatment of any of acute or chronic systematic hypertension, hyperoxia-induced injury, cancer by the pro-apoptotic effect of CO, transplant rejection, post-operative ileus, post-ischemic organ damage, angina, haemorrhagic shock, penile erectile dysfunction, hepatic cirrhosis, cardiac hypertrophy, heart failure and ulcerative colitis or for treatment in balloon angioplasty or aortic transplantation.

49. A method according to claim 45, which is for treatment of any of acute or chronic systemic hypertension, pulmonary hypertension, transplant rejection, post-operative ileus, arteriosclerosis, post-ischemic organ damage, myocardial infarction, penile erectile dysfunction, vascular restenosis, hepatic cirrhosis, cardiac hypertrophy, heart failure, chronic anal fissure, internal anal sphincter disease, anorectal disease, and ulcerative colitis or for treatment in balloon angioplasty or aortic transplantation.

50. A method according to any one of claim 45 wherein the boranocarbonate compound or ion is as defined above.

51. A method according to claim 45 wherein the guanylate cyclase stimulant or stabilizer is a molecule or ion uncombined with the boranocarbonate compound or ion.

52. A method according to claim 45 wherein the guanylate cyclase stimulant or stabilizer is YC-1.

53. A pharmaceutical composition comprising:
a) a boranocarbonate compound or ion which makes available CO suitable for physiological effect; and
b) a guanylate cyclase stimulant or stabiliser.

54. A composition according to claim 53 wherein the boranocarbonate compound or ion is as defined above.

55. A composition according to claim 53 wherein the guanylate cyclase stimulant or stabilizer is a molecule or ion uncombined with the boranocarbonate compound or ion.

56. A composition according to claim 53 wherein the guanylate cyclase stimulant or stabilizer is YC-1.

57. A composition according to claim 53, adapted for one of simultaneous and sequential administration of the boranocarbonate compound or ion and the guanylate cyclase stimulant or stabilizer.
